(19) United States
(12) Patent Application Publication
(10) Pub. No.: US 2026/0108440 A1
(43) Pub. Date: Apr. 23, 2026

(54) Personal Care Compositions

(71) Applicant: Colgate-Palmolive Company, New York, NY (US)

(72) Inventors: Viktor DUBOVOY, Basking Ridge, NJ (US); Ruchir PARIKH, East Windsor, NJ (US); Rahul PATEL, Hillsborough, NJ (US); Xiang CHEN, Somerset, NJ (US); Haroon A. CHAUDHRY, Monroe Township, NJ (US); Zilma Pereira MUNEESWARAN, Highland Park, NJ (US)

(73) Assignee: Colgate-Palmolive Company, New York, NY (US)

(21) Appl. No.: 19/117,293

(22) PCT Filed: Dec. 8, 2023
(86) PCT No.: PCT/US2023/083062

§ 371 Date: Mar. 31, 2025

Related U.S. Application Data
(60) Provisional application No. 63/431,476, filed on Dec. 9, 2022.
(60) Provisional application No. 63/431,479, filed on Dec. 9, 2022.
(60) Provisional application No. 63/431,485, filed on Dec. 9, 2022.

(51) Int. Cl. A61K 8/365; A61K 8/34; A61K 8/35; A61K 8/368; A61K 8/41; A61K 8/42; A61Q 19/10

(52) U.S. Cl. CPC A61K 8/365; A61K 8/345; A61K 8/35; A61K 8/368; A61K 8/416; A61K 8/42; A61Q 19/10; A61K 2800/596; A61K 2800/805

(57) ABSTRACT

Personal care compositions having a surfactant system comprising a cationic surfactant, an amphoteric surfactant, and a non-ionic surfactant; and a preservative system comprising lactic acid, benzalkonium chloride, and at least one of sodium benzoate, potassium sorbate, and a combination of thereof are disclosed herein.

### CROSS-REFERENCE TO RELATED APPLICATIONS

This application claims the benefit of priority from U.S. Provisional Application Nos. 63/431,485; 63/431,479; and 63/431,476, filed Dec. 9, 2022, the contents of which are hereby incorporated by reference herein in their entireties.

### BACKGROUND

Liquid cleansing compositions, such as hand soaps, body wash, etc., such as those in a filled hand soap dispenser have a large concentration of water, which contributes to the overall weight of the product. The weight of the product and size of the dispenser or container adds to the manufacturing, packaging costs, and shipping costs. Since clean water is available readily in most of the world, and packaging can be re-used, it is wasteful to ship water in the liquid cleansers from one location to another and to use the original packages only once.

Liquid cleansing compositions have a viscosity that is generally thicker than other cleansers and as a result have a desirable feel (lubricity) to the user when applied to their skin. Typically, adding water to a concentrate results in a diluted, thinner, i.e., reduced viscosity solution. It is a challenge to develop a concentrated cleansing composition in a delivery vehicle that is easily soluble in water, achieves a viscosity desirable for such cleansing compositions upon dilution with water, and further achieves desirable amounts of foam upon dilution with water.

Additionally, the main functional ingredients in cleansing compositions are surfactants. Surfactants interact with water, thereby allowing it to “wet” surfaces more efficiently. The surfactant-water combination is then able to surround specks of dirt and carry them away with rinsing. Agitation of the water solution, for example by rubbing hands together during washing or lathering also aids the process of removing dirt.

Conventional cleansing compositions such as hand soaps contain surfactants in various amounts. Anionic surfactants are typically included because they provide both cleansing and foaming benefits to a composition. Nonionic surfactants may also be included to provide cleansing, solubilizing, and dispersing properties, and are usually less irritating than anionic surfactants. Nonionic surfactants, however, often exhibit less foaming ability and do not provide any enhancement to viscosity (e.g., oftentimes the composition is thinner and runnier with increased amounts of nonionic surfactants).

The development of cleansing compositions has been driven by a need for certain performance properties that consumers find desirable. For example, consumers often desire cleansing compositions, such as hand soaps, to have an antibacterial effect. However, it can be difficult to obtain cleansing compositions having antibacterial properties and desirable foam and cleansing characteristics. It is therefore difficult to achieve a perfect balance of desirable performance properties.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

In accordance with another aspect, provided is a personal care composition having from about 30 to about 95 wt. % of a surfactant system comprising from about 15 to about 70 wt. % of a cationic surfactant, from about 10 to about 55 wt. % an amphoteric surfactant, and a non-ionic surfactant; and a preservative system comprising at least one of lactic acid, benzalkonium chloride, or a combination thereof, and at least one of sodium benzoate, potassium sorbate, hydroxyacetophenone, ethylhexylglycerin, or a combination of thereof, wherein all weight percentages are based on the total weight of the personal care composition.

In accordance with a further aspect, provided is a diluted personal care composition having a surfactant system comprising a cationic surfactant, an amphoteric surfactant, and a non-ionic surfactant; and from about 0.3 to about 3 wt. % of a preservative system comprising lactic acid, benzalkonium chloride, and least one of sodium benzoate, potassium sorbate, hydroxyacetophenone, ethylhexylglycerin, and a combination of thereof, wherein the personal care composition has a Micro Robustness Index value of greater than 0.75, and all weight percentages are based on the total weight of the diluted personal care composition.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to and can be employed in other apparatuses and methods. Before explaining the disclosed embodiments of the present invention in detail, it is to be understood that the invention is not limited in its application to the details of any particular embodiment disclosed herein. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the personal care composition. The symbol “°” refers to a degree, such as a temperature degree or a degree of an angle. The symbols “h”, “min”, “mL”, “nm”, and “μm” refer to hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

When referring to chemical structures, and names, the symbols “C”. “H”, and “O” mean carbon, hydrogen, and oxygen, respectively. The symbols “−”, “=” and “≡” mean single bond, double bond, and triple bond, respectively.

“Volatile”, as used herein, means having a flash point of less than about 100° C. “Non-volatile”, as used herein, means having a flash point of greater than about 100° C.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

The phrases, “a mixture thereof,” “a combination thereof,” or a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the personal care compositions of the present disclosure can be free or essentially free of all components and elements positively recited throughout the present disclosure. In some instances, the personal care compositions of the present disclosure may be substantially free of non-incidental amounts of the ingredient(s) or compound(s) described herein. A non-incidental amount of an ingredient or compound is the amount of that ingredient or compound that is added into the personal care composition by itself. For example, a personal care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the personal care composition includes both components (or the composition includes more than two components that overlap), an overlapping compound does not represent more than one component. For example, certain compounds may be characterized as both a surfactant and an emulsifier. If a particular personal care composition includes both a surfactant and an emulsifier, a compound that may be characterized as both a surfactant and an emulsifier will serve only as either a surfactant or an emulsifier not both.

For readability purposes, the chemical functional groups are in their adjective form; for each of the adjectives, the word “group” is assumed. For example, the adjective “alkyl” without a noun thereafter, should be read as “an alkyl group.”

Aspects of the invention are directed to personal care compositions and, for instance, personal care compositions having a concentrated formulation. The personal care compositions may be in a form of a concentrated personal care composition that can be diluted to produce a personal care composition in a ready-to-use form. Preferably, the personal care compositions, particularly when in the diluted and/or ready-to-use form, have an antibacterial property.

The inventors discovered that personal care compositions can be formulated with certain surfactant systems and preservative systems to produce a personal care composition in a concentrated form that provides excellent antibacterial properties and foam characteristics upon dilution. Without being limited to any specific theories, the inventors believe that certain combination of ingredients in particular ratios and amounts enables personal care compositions (e.g., in a concentrated/non-diluted form) to have a high concentration of surfactants that are stably solubilized while maintaining desirable rheological properties and providing excellent antibacterial properties upon dilution to produce a diluted and/or ready-to-use form. Preferably, the personal care compositions are formulated to have less than about 1 ppm of 1,4 dioxane.

In accordance with one aspect, provided is a personal care composition having from about 30 to about 95 wt. % of a surfactant system comprising a cationic surfactant, an amphoteric surfactant, and a non-ionic surfactant; and a preservative system comprising lactic acid, benzalkonium chloride, and at least one of sodium benzoate, potassium sorbate, and a combination of thereof, wherein all weight percentages are based on the total weight of the personal care composition.

In accordance with another aspect, provided is a personal care composition having from about 30 to about 95 wt. % of a surfactant system comprising from about 15 to about 70 wt. % of a cationic surfactant, from about 10 to about 55 wt. % an amphoteric surfactant, and a non-ionic surfactant; and a preservative system comprising at least one of lactic acid, benzalkonium chloride, or a combination thereof, and at least one of sodium benzoate, potassium sorbate, hydroxyacetophenone, ethylhexylglycerin, or a combination of thereof, wherein all weight percentages are based on the total weight of the personal care composition.

In accordance with a further aspect, provided is a diluted personal care composition having a surfactant system comprising a cationic surfactant, an amphoteric surfactant, and a non-ionic surfactant; and from about 0.3 to about 3 wt. % of a preservative system comprising lactic acid, benzalkonium chloride, and least one of sodium benzoate, potassium sorbate, hydroxyacetophenone, ethylhexylglycerin, and a combination of thereof, wherein the personal care composition has a Micro Robustness Index value of greater than 0.75, and all weight percentages are based on the total weight of the diluted personal care composition.

The personal care compositions may be formulated to have surfactant systems that comprise about 5 wt. % or less of anionic surfactants, based on the total weight of the personal care composition. For example, the personal care composition contains anionic surfactant(s) in an amount of about 4 wt. % or less, about 3 wt. % or less, about 2 wt. % or less, about 1 wt. % or less, about 0.5 wt. % or less, or about 0.1 wt. % or less, based on the total weight of the personal care composition. In some instances, the personal care composition is substantially free or free of anionic surfactants.

Additionally or alternatively, the personal care compositions may have a limited amount of water. For instance, the personal care compositions may have about 5 wt. % or less of water, based on the total weight of the personal care composition. In some embodiments, the amount of water in the personal care composition is about 4 wt. % or less, about 3 wt. % or less, about 2 wt. % or less, about 1 wt. % or less, or about 0.5 wt. % or less, based on the total weight of the personal care composition. Although the personal care compositions typically include an amount of water, in some embodiments, the personal care composition is substantially free or free of water.

The personal care compositions preferably have less than about 1 ppm of 1,4 dioxane. In some embodiments, the personal care compositions have 1 ppm or less, about 0.7 ppm or less, about 0.5 ppm or less, about 0.3 ppm or less, or about 0.1 ppm or less of 1,4 dioxane.

The personal care compositions may be formulated to have a viscosity of about 100 to about 1,500 cP using a Brookfield DV2T viscometer with a spindle number 5 at 20 RPM and a temperature of 25° C. in a non-diluted form. In some instances, the personal care composition has a viscosity of from about 100 to about 1,500 cP, about 100 to about 1,300 cP, about 100 to about 1,100 cP, about 100 to about 900 cP, about 100 to about 700 cP, about 100 to about 600 cP, about 100 to about 500 cP; from about 250 to about 1,500 cP, about 250 to about 1,300 cP, about 250 to about 1.100 cP, about 250 to about 900 cP, about 250 to about 700 cP, about 250 to about 600 cP, about 250 to about 500 cP; from about 350 to about 1,500 cP, about 350 to about 1,300 cP, about 350 to about 1,100 cP, about 350 to about 900 cP, about 350 to about 700 cP, about 350 to about 600 cP, about 350 to about 500 cP; from about 450 to about 1,500 cP, about 450 to about 1,300 cP, about 450 to about 1,100 cP, about 450 to about 900 cP, about 450 to about 700 cP, about 450 to about 600 cP; from about 550 to about 1,500 cP, about 550 to about 1,300 cP, about 550 to about 1,100 cP, about 550 to about 900 cP, about 550 to about 700 cP, or any range or subrange thereof using a Brookfield DV2T viscometer with a spindle number 5 at 20 RPM and a temperature of 25° C.

The personal care compositions may have a viscosity when in a non-diluted and/or concentrated form that is different from the viscosity of the personal care composition in a diluted and/or ready-to-use form. For example, the viscosity of the personal care composition in the diluted form may be greater than the viscosity of the personal care composition prior to dilution in the concentrated form. In some embodiments, the viscosity of the personal care composition in a diluted form is from about 1,000 cP to about 9,000 cP using a Brookfield DV2T viscometer with a spindle number 5 at 20 RPM and a temperature of 25° C. For instance, when diluted, the personal care composition may have a viscosity of about 1,000 to about 9,000 cP, about 1,000 to about 8,000 cP, about 1,000 to about 7,000 cP, about 1.000 to about 6,000 cP, about 1,000 to about 5,500 cP, about 1,000 to about 5,000 cP, about 1,000 to about 4,700 cP, about 1,000 to about 4,400 cP, about 1,000 to about 4,100 cP; from about 2,000 to about 9,000 cP, about 2,000 to about 8,000 cP, about 2,000 to about 7,000 cP, about 2,000 to about 6,000 cP, about 2,000 to about 5,500 cP, about 2,000 to about 5,000 cP, about 2,000 to about 4,700 cP, about 2,000 to about 4,400 cP, about 2,000 to about 4,100 cP; from about 3,000 to about 9,000 cP, about 3,000 to about 8,000 cP, about 3,000 to about 7,000 cP, about 3,000 to about 6,000 cP, about 3,000 to about 5,500 cP, about 3,000 to about 5,000 cP, about 3,000 to about 4,700 cP, about 3,000 to about 4,400 cP, about 3,000 to about 4,100 cP; from about 3,500 to about 9,000 cP, about 3,500 to about 8,000 cP, about 3,500 to about 7,000 cP, about 3,500 to about 6,000 cP, about 3,500 to about 5,500 cP, about 3,500 to about 5,000 cP, about 3,500 to about 4,700 cP, about 3,500 to about 4,400 cP, about 3,500 to about 4.100 cP; from about 3,800 to about 9,000 cP, about 3,800 to about 8,000 cP, about 3,800 to about 7.000 cP, about 3,800 to about 6,000 cP, about 3,800 to about 5,500 cP, about 3,800 to about 5,000 cP, about 3,800 to about 4,700 cP, about 3,800 to about 4,400 cP, about 3,800 to about 4,100 cP; from about 4,100 to about 9,000 cP, about 4,100 to about 8,000 cP, about 4,100 to about 7,000 cP, about 4,100 to about 6,000 cP, about 4,100 to about 5,500 cP, about 4,100 to about 5,000 cP, about 4,100 to about 4,700 cP, about 4,100 to about 4,400 cP, or any range or subrange thereof, using a Brookfield DV2T viscometer with a spindle number 5 at 20 RPM and a temperature of 25° C.

Suitable components, such as those listed below, may be included or excluded from the formulations for the personal care compositions depending on the specific combination of other components, the form of the personal care compositions, and/or the use of the formulation (e.g., a diluted/ready-to-use personal care composition, a cleansing composition, an antibacterial soap, etc.). According to certain aspects of the invention, the personal care composition is in the form of a concentrated personal care composition encapsulated in a pod. In some embodiments, the personal care composition is a cleansing composition in the form of a hand soap and/or a body wash.

The personal care compositions may include a surfactant system in an amount that may vary, but generally is in the range of from about 30 to about 95 wt. %, based on the total weight of the personal care composition. For example, the personal care composition may include a surfactant system in an amount from about 30 to about 90 wt. %, about 30 to about 85 wt. %, about 30 to about 80 wt. %, about 30 to about 75 wt. %, about 30 to about 70 wt. %, about 30 to about 65 wt. %, about 30 to about 60 wt. %, about 30 to about 55 wt. %; from about 40 to about 95 wt. %, about 40 to about 90 wt. %, about 40 to about 85 wt. %, about 40 to about 80 wt. %, about 40 to about 75 wt. %, about 40 to about 70 wt. %, about 40 to about 65 wt. %, about 40 to about 60 wt. %, about 40 to about 55 wt. %; from about 48 to about 95 wt. %, about 48 to about 90 wt. %, about 48 to about 85 wt. %, about 48 to about 80 wt. %, about 48 to about 75 wt. %, about 48 to about 70 wt. %, about 48 to about 65 wt. %, about 48 to about 60 wt. %, about 48 to about 55 wt. %; from about 52 to about 95 wt. %, about 52 to about 90 wt. %, about 52 to about 85 wt. %, about 52 to about 80 wt. %, about 52 to about 75 wt. %, about 52 to about 70 wt. %, about 52 to about 65 wt. %, about 52 to about 60 wt. %; from about 56 to about 95 wt. %, about 56 to about 90 wt. %, about 56 to about 85 wt. %, about 56 to about 80 wt. %, about 56 to about 75 wt. %, about 56 to about 70 wt. %, about 56 to about 65 wt. %; from about 60 to about 95 wt. %, about 60 to about 90 wt. %, about 60 to about 85 wt. %, about 60 to about 80 wt. %, about 60 to about 75 wt. %, about 60 to about 70 wt. %, about 60 to about 65 wt. %; from about 66 to about 95 wt. %, about 66 to about 90 wt. %, about 66 to about 85 wt. %, about 66 to about 80 wt. %, about 66 to about 75 wt. %, about 66 to about 70 wt. %; from about 70 to about 95 wt. %, about 70 to about 90 wt. %, about 70 to about 85 wt. %, about 70 to about 80 wt. %; from about 73 to about 95 wt. %, about 73 to about 90 wt. %, about 73 to about 85 wt. %, about 73 to about 80 wt. %, or any range or subrange thereof, based on the total weight of the personal care composition.

The surfactant systems disclosed herein typically comprise two or more surfactants selected from cationic surfactants, nonionic surfactants, amphoteric surfactants, zwitterionic surfactants, and combinations of two or more thereof. For example, the surfactant system may include two, three, four, five, six, seven, eight, or nine or more surfactants. In some embodiments, the surfactant system comprises from two to eight, from two to seven, from two to six, from two to five, from two to four, from three to seven, from three to six, from three to five, from four to eight, four to seven, four to six, or any range or subrange thereof. Preferably, the surfactant system may include two or more surfactants, with the two or more surfactants being chosen from different categories of cationic surfactants, nonionic surfactants, amphoteric surfactants, and zwitterionic surfactants. For example, the surfactant system may comprise at least one cationic surfactant and at least one amphoteric surfactant or may comprise at least one cationic surfactant and at least one nonionic surfactant. In some instances, the surfactant system may comprises at least one cationic surfactant, at least one amphoteric surfactant, and at least one nonionic surfactant.

The surfactant system of the personal care composition typically includes one or more cationic surfactant(s) in an amount of, e.g., from about 15 to about 70 wt. %, based on the total weight of the personal care composition. For instance, the total amount of cationic surfactant(s) in the personal care composition may be from about 15 to about 65 wt. %, about 15 to about 60 wt. %, about 15 to about 55 wt. %, about 15 to about 52 wt. %, about 15 to about 49 wt. %, about 15 to about 46 wt. %, about 15 to about 43 wt. %, about 15 to about 39, about 15 to about 36 wt. %; from about 25 to about 70 wt. %, about 25 to about 65 wt. %, about 25 to about 60 wt. %, about 25 to about 55 wt. %, about 25 to about 52 wt. %, about 25 to about 49 wt. %, about 25 to about 46 wt. %, about 25 to about 43 wt. %, about 25 to about 39, about 25 to about 36 wt. %; from about 30 to about 70 wt. %, about 30 to about 65 wt. %, about 30 to about 60 wt. %, about 30 to about 55 wt. %, about 30 to about 52 wt. %, about 30 to about 49 wt. %, about 30 to about 46 wt. %, about 30 to about 43 wt. %, about 30 to about 39 wt. %; from about 33 to about 70 wt. %, about 33 to about 65 wt. %, about 33 to about 60 wt. %, about 33 to about 55 wt. %, about 33 to about 52 wt. %, about 33 to about 49 wt. %, about 33 to about 46 wt. %, about 33 to about 43 wt. %, about 33 to about 39 wt. %; from about 36 to about 70 wt. %, about 36 to about 65 wt. %, about 36 to about 60 wt. %, about 36 to about 55 wt. %, about 36 to about 52 wt. %, about 36 to about 49 wt. %, about 36 to about 46 wt. %, about 36 to about 43 wt. %; from about 39 to about 70 wt. %, about 39 to about 65 wt. %, about 39 to about 60 wt. %, about 39 to about 55 wt. %, about 39 to about 52 wt. %, about 39 to about 49 wt. %, about 39 to about 46 wt. %; from about 42 to about 70 wt. %, about 42 to about 65 wt. %, about 42 to about 60 wt. %, about 42 to about 55 wt. %, about 42 to about 52 wt. %, about 42 to about 49 wt. %; from about 45 to about 70 wt. %, about 45 to about 65 wt. %, about 45 to about 60 wt. %, about 45 to about 55 wt. %, or any range or subrange thereof, based on the total weight of the personal care composition.

The term “cationic surfactant” means a surfactant that may be positively charged when it is contained in the personal care compositions according to the disclosure. This surfactant may bear one or more positive permanent charges or may contain one or more functional groups that are cationizable in the personal care composition according to the disclosure.

The one or more cationic surfactants, if present, may include or be chosen from quaternary ammonium compounds, amidoamines, or a mixture thereof. Examples of cationic surfactants that may be suitable for the personal care composition include or may be chosen from cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, olcyl hydroxyethyl imidazoline, stearamidopropyldimethylamine, behenamidopropyldimethylamine, behenamidoethyldimethylamine, arachidamidopropyldimethylamine, arachidamido-propyidicthylaminc, arachidamidocthyidicthylamine, arachidamidocthyidimcthylaminc, and a combination of two or more thereof.

Additional, non-limiting examples of cationic surfactants include: cetyl trimethyl ammonium chloride available, for example, with trade name CA-2350 from Nikko Chemicals and CTAC 30KC available from KCl, stearyl trimethyl ammonium chloride with trade name Arquad 18/50 available from Akzo Nobel, hydrogenated tallow alkyl trimethyl ammonium chloride, stearyl dimethyl benzyl ammonium chloride, stearyl propyleneglycol phosphate dimethyl ammonium chloride, stearoyl amidopropyl dimethyl benzyl ammonium chloride, stearoyl amidopropyl dimethyl (myristylacetate) ammonium chloride, and N-(stearoyl colamino formyl methy) pyridinium chloride.

Hydrophilically substituted cationic surfactants in which at least one of the substituents contain one or more aromatic, ether, ester, amido, or amino moieties present as substituents or as linkages in the chain may also be in the personal care composition. Non-limiting examples of hydrophilically substituted cationic surfactants include the materials having the following INCI designations: quaternium-16, quaternium-26, quaternium-27, quaternium-30, quaternium-33, quaternium-43, quaternium-52, quaternium-53, quaternium-56, quaternium-60, quaternium-61, quaternium-62, quaternium-70, quaternium-71, quaternium-72, quaternium-75, quaternium-76 hydrolyzed collagen, quaternium-77, quaternium-78, quaternium-79 hydrolyzed collagen, quaternium-79 hydrolyzed keratin, quaternium-79 hydrolyzed milk protein, quaternium-79 hydrolyzed silk, quaternium-79 hydrolyzed soy protein, and quaternium-79 hydrolyzed wheat protein, quaternium-80, quaternium-81, quaternium-82, quaternium-83, quaternium-84, or a combination of two or more thereof.

In certain instances, the cationic surfactant is selected from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidoethyldimethylamine, arachidamidopropyldimethylamine, arachidamido-propyidicthylaminc, arachidamidocthyidicthylaminc, arachidamidocthyidimcthylaminc, lauramidopropyl dimethylamine, myristamidopropyl dimethylamine, dilinolcamidopropyl dimethylamine, palmitamidopropyl dimethylamine, and a combination of two or more thereof. In some cases, the cationic surfactant is selected from cetrimonium chloride, behentrimonium chloride, and a combination of two or more thereof.

The personal care composition may include one or more amphoteric surfactant(s) in an amount that may vary, but typically ranges from about 10 to about 55 wt. %, based on the total weight of the personal care composition. For example, the total amount of amphoteric surfactant(s) in the personal care composition may be from about 10 to about 50 wt. %, about 10 to about 45 wt. %, about 10 to about 42 wt. %, about 10 to about 39 wt. %, about 10 to about 36 wt. %, about 10 to about 34 wt. %, about 10 to about 32 wt. %; from about 20 to about 55 wt. %, about 20 to about 50 wt. %, about 20 to about 45 wt. %, about 20 to about 42 wt. %, about 20 to about 39 wt. %, about 20 to about 36 wt. %, about 20 to about 34 wt. %, about 20 to about 32 wt. %; from about 25 to about 55 wt. %, about 25 to about 50 wt. %, about 25 to about 45 wt. %, about 25 to about 42 wt. %, about 25 to about 39 wt. %, about 25 to about 36 wt. %, about 25 to about 34 wt. %, about 25 to about 32 wt. %; from about 29 to about 55 wt. %, about 29 to about 50 wt. %, about 29 to about 45 wt. %, about 29 to about 42 wt. %, about 29 to about 39 wt. %, about 29 to about 36 wt. %, about 29 to about 34 wt. %; from about 34 to about 55 wt. %, about 34 to about 50 wt. %, about 34 to about 45 wt. %, about 34 to about 42 wt. %, about 34 to about 39 wt. %; from about 38 to about 55 wt. %, about 38 to about 50 wt. %, about 38 to about 45 wt. %, about 38 to about 42 wt. %; from about 42 to about 55 wt. %, about 42 to about 50 wt. %, about 42 to about 45 wt. %; from about 46 to about 55 wt. %, about 46 to about 50 wt. %; from about 50 to about 55 wt. %, or any range or subrange thereof, based on the total weight of the personal care composition.

Amphoteric surfactants are typically characterized by a combination of high surfactant activity, lather forming and mildness. The amphoteric surfactant may comprise a substituent containing 8 to 18 carbon atoms and a substituent containing one or more carboxylate, sulfonate, sulfate, phosphate, or phosphonate. For instance, the amphoteric surfactant may have an alkyl group comprising from 8 to 20 carbon atoms, 8 to 16 carbon atoms, 10 to 16 carbon atoms, or 10 to 13 carbon atoms. The amphoteric surfactant(s) may include, but are not limited to, derivatives of aliphatic secondary and tertiary amines in which the aliphatic radical can be straight chain or branched and wherein one of the aliphatic substituents contains about 8 to about 18 carbon atoms and one contains an anionic water solubilizing group, e.g., carboxy, sulfonate, sulfate, phosphate, or phosphonate.

The amphoteric surfactants may include alkyl amphoproprionates, betaines, alkyl sultaines, alkyl amphoacetates, and mixtures thereof. In some instances, the personal care composition includes one or more alkyl amphopropionates. Non-limiting examples of alkyl amphopropionates include cocoamphopropionate, cornamphopropionate, caprylamphopropionate, cornamphopropionate, caproamphopropionate, oleoamphopropionate, isostearoamphopropionate, stearoamphopropionate, lauroamphopropionate, salts thereof, and a mixture thereof. In one embodiment, the alkyl amphopropionate comprises sodium cocoamphopropionate.

The personal care composition may include an amphoteric surfactant selected from betaine surfactants (also referred to as betaines). Examples of amphoteric surfactants include, but are not limited to, a range of betaines including, for example, high alkyl betaines, such as coco dimethyl carboxymethyl betaine, lauryl dimethyl carboxy-methyl betaine, lauryl dimethyl alpha-carboxyethyl betaine, cetyl dimethyl carboxymethyl betaine, lauryl bis-(2-hydroxyethyl) carboxy methyl betaine, stearyl bis-(2-hydroxypropyl) carboxymethyl betaine, oleyl dimethyl gamma-carboxypropyl betaine, and lauryl bis-(2-hydroxypropyl) alpha-carboxyethyl betaine, sulfobetaines such as coco dimethyl sulfopropyl betaine, stearyl dimethyl sulfopropyl betaine, amido betaines, amidosulfobetaines and the like. Preferably, a betaine surfactant is selected from coca betaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, and combinations of two or more thereof. In some embodiments, at least one betaine surfactant is selected from coco betaine, cocamidopropyl betaine, behenyl betaine, capryl/capramidopropyl betaine, and lauryl betaine, and a combination of two or more thereof. In further embodiments, the betaine surfactant is selected from cocamidopropyl betaine, lauryl dimethyl betaine, cetyl dimethyl betaine, and cocoamphodiacetate, and a combination of two or more thereof. In at least one embodiment, the personal care composition comprises coco betaine, cocamidopropyl betaine, or a combination thereof.

The amphoteric surfactant may, in some cases, be selected sultaines, also known as sulfobetaines, and hydroxy sultaines, for example, cocamidopropyl hydroxysultain. The sultaines may be an alkyl sultaines having from 6-20 carbons, 8-18 carbons, 8-16 carbons, or 10-16 carbons. Non-limiting examples of alkyl sultaincs include hydroxyl sultaincs having a structure according to the following formula:

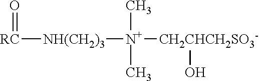

wherein R is an alkyl group having 8-18 carbon atoms. Examples of alkyl sultaine surfacants include, but are not limited to, cocamidopropyl hydroxysultaine, lauryl hydroxysultaine, and a combination of two or more thereof.

In some embodiments, the surfactant system includes an amphoteric surfactant selected from betaine surfactants and, optionally, an additional amphoteric surfactant other than betaines including those discussed above. The one or more betaine surfactants may, preferably, be selected from coca betaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, and a combination of two or more thereof.

The personal care compositions may include one or more nonionic surfactant(s), e.g., in an amount from about 1 to about 25 wt. %, based on the total weight of the personal care composition. For example, the total amount of nonionic surfactant(s) in the personal care composition may be from about 1 to about 20 wt. %, about 1 to about 16 wt. %, about 1 to about 12 wt. %, about 1 to about 10 wt. %, about 1 to about 8 wt. %, about 1 to about 7 wt. %, about 1 to about 6 wt. %, about 1 to about 5 wt. %, about 1 to about 4 wt. %, about 1 to about 3 wt. %; from about 2 to about 25 wt. %, about 2 to about 20 wt. %, about 2 to about 16 wt. %, about 2 to about 12 wt. %, about 2 to about 10 wt. %, about 2 to about 8 wt. %, about 2 to about 7 wt. %, about 2 to about 6 wt. %, about 2 to about 5 wt. %, about 2 to about 4 wt. %, about 2 to about 3 wt. %; from about 3 to about 25 wt. %, about 3 to about 20 wt. %, about 3 to about 16 wt. %, about 3 to about 12 wt. %, about 3 to about 10 wt. %, about 3 to about 8 wt. %, about 3 to about 7 wt. %, about 3 to about 6 wt. %, about 3 to about 5 wt. %, about 3 to about 4 wt. %; from about 4 to about 25 wt. %, about 4 to about 20 wt. %, about 4 to about 16 wt. %, about 4 to about 12 wt. %, about 4 to about 10 wt. %, about 4 to about 8 wt. %, about 4 to about 7 wt. %, about 4 to about 6 wt. %; from about 6 to about 25 wt. %, about 6 to about 20 wt. %, about 6 to about 16 wt. %, about 6 to about 12 wt. %, about 6 to about 10 wt. %; from about 8 to about 25 wt. %, about 8 to about 20 wt. %, about 8 to about 16 wt. %, about 8 to about 12 wt. %, or any range or subrange thereof, based on the total weight of the personal care composition.

The nonionic surfactant is selected from alkyl polyglucosides, alkylamides, alkanolamides, polyoxyalkylenated nonionic surfactants, polyglycerolated nonionic surfactants, ethoxylated fatty esters (preferably, polyglyceryl-2-caprate), and a combination of two or more thereof. The nonionic surfactant, such as alkyl polyglucosides, alkylamides, and alkanolamides, may have an alkyl group having 8 to 32 carbons, 8 to 26 carbons, 8 to 22 carbons, 8 to 18 carbons, 10 to 22 carbons, 10 to 18 carbons, 12 to 22 carbons, or 12 to 18 carbons. In at least one preferred embodiment, the nonionic surfactant is an alkyl polyglucoside, an alkylamide, or an alkanolamide having an alkyl group of 12 to 18 carbons.

Examples of alkanolamides include fatty acid alkanolamides. The fatty acid alkanolamides may be fatty acid monoalkanolamides or fatty acid dialkanolamides or fatty acid isoalkanolamides, and may have a C₂₋₈ hydroxyalkyl group (the C₂₋₈ chain can be substituted with one or more-OH group). Preferably, the fatty acid alkanolamides are chosen from fatty acid diethanolamides (DEA) or fatty acid monocthanolamides (MEA), fatty acid monoisopropanolamides (MIPA), fatty acid diisopropanolamides (DIPA), and fatty acid glucamides (acyl glucamides). In some preferred embodiments, the nonionic surfactant comprises cocamide monoethanolamine (MEA), cocamide diethanolamine (DEA) or cocamide diisopropanolamine (DIPA), or a combination of two or more thereof. In at least one embodiment, the nonionic surfactant comprises cocamide DIPA.

Additional examples of fatty acid alkanolamides include oleic acid diethanolamide, myristic acid monoethanolamide, soya fatty acids diethanolamide, stearic acid ethanolamide, oleic acid monoisopropanolamide, linoleic acid diethanolamide, stearic acid monocthanolamide (Stearamide MEA), behenic acid monoethanolamide, isostearic acid monoisopropanolamide (isostearamide MIPA), erucic acid diethanolamide, ricinoleic acid monocthanolamide, coconut fatty acid monoisopropanolamide (cocoamide MIPA), coconut acid monoethanolamide (Cocamide MEA), palm kernel fatty acid diethanolamide, coconut fatty acid diethanolamide, lauric diethanolamide, polyoxyethylene coconut fatty acid monocthanolamide, coconut fatty acid monoethanolamide, lauric monoethanolamide, lauric acid monoisopropanolamide (lauramide MIPA), myristic acid monoisopropanolamide (Myristamide MIPA), coconut fatty acid diisopropanolamide (cocamide DIPA), and mixtures thereof. In some embodiments, the nonionic surfactant(s) comprises Cocamide MEA, an alkylamide, having an alkyl group of 12 to 18 carbons, or a combination of two or more thereof.

The nonionic surfactant may be selected from alkyl polyglucosides in some cases. In some instances, the alkyl polyglucoside(s) are chosen from lauryl glucoside, octyl glucoside, decyl glucoside, coco glucoside, caprylyl/capryl glucoside, and sodium lauryl glucose carboxylate, and a combination of two or more thereof. In at least one preferred embodiment, the personal care composition includes one or more nonionic surfactant comprising caprylyl glucoside and/or caprylyl/capryl glucoside. Non-limiting examples of nonionic surfactants worth mentioning include esters of polyols with fatty acids with a saturated or unsaturated chain containing for example from 8 to 24 carbon atoms, preferably 12 to 22 carbon atoms, and alkoxylated derivatives thereof. For example, the personal care composition may comprise polyethylene glycol esters of a C₈-C₂₄, such as C₁₂-C₂₂ glycol esters; sorbitol esters of a C₈-C₂₄, such as C₁₂-C₂₂ sorbitol esters; sugar (sucrose, glucose, alkylglycose) esters of a C₈-C₂₄; ethers of sugar and a C₈-C₂₄; or combinations of two or more thereof.

The nonionic surfactant(s) may be selected from ethoxylated fatty esters, such as adducts of ethylene oxide with esters of lauric acid, palmitic acid, stearic acid or behenic acid, and mixtures thereof, including those containing from 9 to 100 oxyethylene groups. For example, the ethoxylated fatty esters may comprise PEG-9 laurate to PEG-50 laurate; PEG-9 palmitate to PEG-50 palmitate; PEG-9 stearate to PEG-50 stearate; PEG-9 palmitostearate to PEG-50 palmitostearate; PEG-9 behenate to PEG-50 behenate; polyethylene glycol 100 EO monostearate (such as, PEG-100 stearate); and combinations of two or more thereof. As examples of glyceryl esters of fatty acids, glyceryl stearate (glyceryl mono-, di- and/or tristearate), glyceryl ricinoleate, and a combination of two or more thereof may be incorporated in the personal care composition. Examples of glyceryl esters of C₈-C₂₄ alkoxylated fatty acids include polyethoxylated glyceryl stearate (glyceryl mono-, di- and/or tristearate), such as PEG-20 glyceryl stearate.

The personal composition may, in some instance, include one or more anionic surfactant(s). For example, the personal care composition may include from about 0.1 wt. %, from about 0.5 wt. %, from about 1 wt. %, from about 2 wt. %, from about 3 wt. %, or from about 3.5 wt. % up to about 1 wt. %, up to about 2 wt. %, up to about 3 wt. %, up to about 4 wt. %, or up to about 5 wt. %, including any range or subrange formed therefrom, of anionic surfactant, based on the total weight of the personal care composition. Although some embodiments may include anionic surfactants, the personal care compositions may have anionic surfactants in an amount of about 5 wt. % or less, about 4 wt. % or less, about 3 wt. % or less, about 2 wt. % or less, about 1 wt. % or less, about 0.5 wt. % or less, or about 0.1 wt. % or less of a cationic surfactant, based on the total weight of the personal care composition. In some embodiments, the personal care composition is substantially free or free of anionic surfactants.

Examples of anionic surfactants include from sulfate anionic surfactants, acyl isethionates, acyl amino acids, alkyl sulfonates, alkyl sulfosuccinates, alkyl sulfoacetates, alkoxylated monoacids, salts thereof, and combinations of two or more thereof. Sulfate anionic surfactants worth mentioning include alkyl sulfates and alkyl ether sulfates. The alkyl sulfates and/or alkyl ether sulfates may have an alkyl group comprising 8 to 20 carbon atoms, 8 to 16 carbon atoms, 10 to 16 carbon atoms, or 10 to 13 carbon atoms. Non-limiting examples of alkyl sulfate and alkyl ether sulfate salts include sodium lauryl sulfate (SLS), sodium dodecyl sulfate (SDS), sodium lauryl ether sulfate (SLES), ammonium lauryl sulfate, and ammonium laureth sulfate. For instance, the sulfate anionic surfactants may be chosen from sodium lauryl sulfate, sodium laureth sulfate, a salt thereof, and a combination of two or more thereof.

The personal care compositions may, additionally or alternatively, include or exclude non-sulfate anionic surfactants, such as acyl isethionates, acyl amino acids, alkyl sulfonates, alkyl sulfosuccinates, alkyl sulfoacetates, alkoxylated monoacids, salts thereof, and combinations of two or more thereof. Non-limiting examples of acyl isethionates include sodium iscthionate, sodium cocoyl isethionate, sodium lauroyl methyl isethionate, and sodium cocoyl methyl isethionate.

Acyl amino acids may include amino acid surfactants based on alanine, arginine, aspartic acid, glutamic acid, glycine, isoleucine, leucine, lysine, phenylalanine, serine, tyrosine, valine, sarcosine, threonine, and taurine. The cation associated with the acyl amino acid can be sodium or potassium. Alternatively, the cation can be an organic salt, such as triethanolamine (TEA) or a metal salt.

Non-limiting examples of acyl sarcosinates include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, and ammonium lauroyl sarcosinate. Non-limiting examples of acyl taurate salts include sodium cocoyl taurate, sodium methyl cocoyl taurate, oleoyl taurate. In at least one preferred embodiment, the personal care composition includes one or more anionic surfactant comprising oleoyl taurate and/or a salt thereof.

Non-limiting examples of alkyl sulfosuccinates salts include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, or a combination of two or more thereof.

Non-limiting examples of alkyl sulfacetates includes, e.g., alkyl sulfoacetates such as C₄-C₁₈ fatty alcohol sulfoacetates and/or salts thereof. A preferred sulfoacetate salt is sodium lauryl sulfoacetate. Useful cations for the salts include alkali metal ions such as sodium or potassium, ammonium ions, or alkanolammonium ions such as monoethanolammonium or triethanolammonium ions.

Examples of alkoxylated monoacids include, but are not limited to: butoxynol-5 carboxylic acid, butoxynol-19 carboxylic acid, capryleth-4 carboxylic acid, capryleth-6 carboxylic acid, capryleth-9 carboxylic acid, ceteareth-25 carboxylic acid, coceth-7 carboxylic acid, C₉₋₁₁ pareth-6 carboxylic acid, C₁₁₋₁₅ pareth-7 carboxylic acid, C₁₂₋₁₃ pareth-5 carboxylic acid, C₁₂₋₁₃ pareth-8 carboxylic acid, C₁₂₋₁₃ pareth-12 carboxylic acid, C₁₂₋₁₅ pareth-7 carboxylic acid, C₁₂₋₁₅ pareth-8 carboxylic acid, C₁₄₋₁₅ pareth-8 carboxylic acid, deceth-7 carboxylic acid, laureth-3 carboxylic acid, laureth-4 carboxylic acid, laureth-5 carboxylic acid, laureth-6 carboxylic acid, laureth-8 carboxylic acid, laureth-10 carboxylic acid, laureth-11 carboxylic acid, laureth-12 carboxylic acid, laureth-13 carboxylic acid, laureth-14 carboxylic acid, laureth-17 carboxylic acid, PPG-6-laureth-6 carboxylic acid, ppg-8-steareth-7 carboxylic acid, myreth-3 carboxylic acid, myreth-5 carboxylic acid, nonoxynol-5 carboxylic acid, nonoxynol-8 carboxylic acid, nonoxynol-10 carboxylic acid, octeth-3 carboxylic acid, octoxynol-20 carboxylic acid, oleth-3 carboxylic acid, oleth-6 carboxylic acid, oleth-10 carboxylic acid, ppg-3-deceth-2 carboxylic acid, capryleth-2 carboxylic acid, ceteth-13 carboxylic acid, deceth-2 carboxylic acid, hexeth-4 carboxylic acid, isosteareth-6 carboxylic acid, isosteareth-11 carboxylic acid, trudeceth-3 carboxylic acid, trideceth-6 carboxylic acid, trideceth-8 carboxylic acid, trideceth-12 carboxylic acid, trideceth-3 carboxylic acid, trideceth-4 carboxylic acid, trideceth-7 carboxylic acid, trideceth-15 carboxylic acid, trideceth-19 carboxylic acid, undeceth-5 carboxylic acid and a combination of two or more thereof.

The personal care compositions may be formulated to have a surfactant system having certain weight ratios of surfactants. For example, the personal care composition may be formulated to have a weight ratio of cationic surfactant(s) to amphoteric surfactant(s) ranging from about 4:1 to about 1:2. In some embodiments, the personal care composition may have a weight ratio of the total amount of cationic surfactant(s) to the total amount of the amphoteric surfactant(s) that is from about 3.5:1 to about 1:2, about 3:1 to about 1:2, about 2:1 to about 1:2, about 1:1 to about 1:2; from about 4:1 to about 1:1, about 3:1 to about 1:1, about 2:1 to about 1:1, or any range or subrange formed therefrom.

Additionally or alternatively, the personal care compositions may be formulated to have a surfactant system having a weight ratio of cationic surfactant(s) to non-ionic surfactant(s) ranging from about 30:1 to about 10:1. For instance, the personal care composition may have a weight ratio of the total amount of cationic surfactant(s) to the total amount of non-ionic surfactant(s) of about 30:1 to about 10:1, about 28:1 to about 10:1, about 26:1 to about 10:1, about 25:1 to about 10:1, about 24:1 to about 10:1, about 22:1 to about 10:1, about 20:1 to about 10:1; from about 30:1 to about 12:1, about 28:1 to about 12:1, about 26:1 to about 12:1, about 25:1 to about 12:1, about 24:1 to about 12:1, about 22:1 to about 12:1, about 20:1 to about 12:1; from about 30:1 to about 14:1, about 28:1 to about 14:1, about 26:1 to about 14:1, about 25:1 to about 14:1, about 24:1 to about 14:1, about 22:1 to about 14:1, about 20:1 to about 14:1; from about 30:1 to about 15:1, about 28:1 to about 15:1, about 26:1 to about 15:1, about 25:1 to about 15:1, about 24:1 to about 15:1, about 22:1 to about 15:1, about 20:1 to about 15:1; from about 30:1 to about 17:1, about 28:1 to about 17:1, about 26:1 to about 17:1, about 25:1 to about 17:1, about 24:1 to about 17:1, about 22:1 to about 17:1; from about 30:1 to about 19:1, about 28:1 to about 19:1, about 26:1 to about 19:1, about 25:1 to about 19:1, about 24:1 to about 19:1; from about 30:1 to about 21:1, about 28:1 to about 21:1, about 26:1 to about 21:1, about 25:1 to about 21:1, about 24:1 to about 21:1, about 22:1 to about 21:1, about 20:1 to about 21:1, or any range or subrange formed therefrom.

In addition to the surfactant system, the personal care compositions typically comprise a preservative system. The preservative system may include at least one of lactic acid, benzalkonium chloride, or a combination thereof, and at least one of sodium benzoate, potassium sorbate, hydroxyacetophenone, ethylhexylglycerin, or a combination of thereof. In some embodiments, the preservative system includes lactic acid, benzalkonium chloride, and at least one of sodium benzoate, potassium sorbate, and a combination of thereof.

The preservative system may be present in an amount that may vary, e.g., depending on the desired concentration of the preservative system in the diluted and/or ready-to-use personal care composition upon dilution of the personal care composition. The personal care composition prior to dilution (e.g. in a non-diluted and/or concentrated form) may be formulated to have a preservative system in an amount from about 1 to about 25 wt. %, based on the total weight of the personal care composition. For example, the preservative system may be present in the personal care composition prior to dilution (e.g. in a concentrated form) in an amount from about 1 to about 22 wt. %, about 1 to about 19 wt. %, about 1 to about 16 wt. %, about 1 to about 14 wt. %, about 1 to about 12 wt. %, about 1 to about 10 wt. %, about 1 to about 8 wt. %, about 1 to about 6 wt. %, about 1 to about 4 wt. %, about 1 to about 3 wt. %; from about 2 to about 25 wt. %, about 2 to about 22 wt. %, about 2 to about 19 wt. %, about 2 to about 16 wt. %, about 2 to about 14 wt. %, about 2 to about 12 wt. %, about 2 to about 10 wt. %, about 2 to about 8 wt. %, about 2 to about 6 wt. %, about 2 to about 4 wt. %, about 2 to about 3 wt. %; from about 3 to about 25 wt. %, about 3 to about 22 wt. %, about 3 to about 19 wt. %, about 3 to about 16 wt. %, about 3 to about 14 wt. %, about 3 to about 12 wt. %, about 3 to about 10 wt. %, about 3 to about 8 wt. %, about 3 to about 6 wt. %, about 3 to about 4 wt. %; from about 5 to about 25 wt. %, about 5 to about 22 wt. %, about 5 to about 19 wt. %, about 5 to about 16 wt. %, about 5 to about 14 wt. %, about 5 to about 12 wt. %, about 5 to about 10 wt. %, about 5 to about 8 wt. %; from about 7 to about 25 wt. %, about 7 to about 22 wt. %, about 7 to about 19 wt. %, about 7 to about 16 wt. %, about 7 to about 14 wt. %, about 7 to about 12 wt. %; from about 10 to about 25 wt. %, about 10 to about 22 wt. %, about 10 to about 19 wt. %, about 10 to about 16 wt. %, about 10 to about 14 wt. %; from about 13 to about 25 wt. %, about 13 to about 22 wt. %, about 13 to about 19 wt. %; from about 15 to about 25 wt. %, about 15 to about 22 wt. %, about 15 to about 19 wt. %; from about 17 to about 25 wt. %, about 17 to about 22 wt. %, or any range or subrange thereof, based on the total weight of the personal care composition.

The personal care composition when in a diluted form (also referred herein as diluted personal care composition) and/or ready-to-use form may have a preservative system present in an amount of about 0.3 to about 3 wt. %, based on the total weight of the diluted personal care composition. For example, the amount of the preservative system in the diluted personal care composition may be from about 0.3 to about 3 wt. %, about 0.3 to about 2.5 wt. %, about 0.3 to about 2 wt. %, about 0.3 to about 1.5 wt. %, about 0.3 to about 1 wt. %; from about 0.5 to about 3 wt. %, about 0.5 to about 2.5 wt. %, about 0.5 to about 2 wt. %, about 0.5 to about 1.5 wt. %, about 0.5 to about 1 wt. %; from about 0.8 to about 3 wt. %, about 0.8 to about 2.5 wt. %, about 0.8 to about 2 wt. %, about 0.8 to about 1.5 wt. %; from about 1.1 to about 3 wt. %, about 1.1 to about 2.5 wt. %, about 1.1 to about 2 wt. %, about 1.1 to about 1.5 wt. %; from about 1.5 to about 3 wt. %, about 1.5 to about 2.5 wt. %, about 1.5 to about 2 wt. %; from about 2 to about 3 wt. %, about 2 to about 2.5 wt. %, about 2.5 to about 3 wt. %, or any range or subrange thereof based on the total weight of the diluted personal care composition.

The preservative system typically includes at least one of lactic acid or benzalkonium chloride, and at least one of sodium benzoate, potassium sorbate, hydroxyacetophenone, ethylhexylglycerin, or a combination of thereof. In some embodiments, the preservative system includes lactic acid, benzalkonium chloride, and at least one of sodium benzoate, potassium sorbate, hydroxyacetophenone, ethylhexylglycerin, or a combination of thereof. For example, the preventive system may include a combination of two or more of lactic acid, benzalkonium chloride, sodium benzoate, and potassium sorbate, or combination of two or more of benzalkonium chloride, hydroxyacetophenone, and ethylhexylglycerin. In some preferred embodiments, the preservative system comprises lactic acid, benzalkonium chloride, sodium benzoate, potassium sorbate, benzalkonium chloride, hydroxyacetophenone, ethylhexylglycerin, or a combination of two or more thereof.

Additional preservatives that may incorporated in the preservative system include, but are not limited to, 4-hydroxy benzoic acid, benzoic acid, sorbic acid, dehydroacetic acid, triclosan, benzyl alcohol, chlorophenesin, salicylic acid, hydroxy acetophenone, caprylhydroxamic acid, phenethyl alcohol, p-anisic acid, glyceryl caprylate, levulinic acid, sodium levulinate, propanediol, hexanediol, pentylene glycol, capryloyl glycine, methylpropanediol, phenylpropanol, sodium anisate, or a combination of two or more thereof. The preservative system may, in some cases, include or exclude one or more of the following preservatives: chlorhexidine, chlorhexidine digluconate, chlorhexidine dihydrochloride, chlorhexidine diacetate, chlorhexidine gluconate, chlorhexidine hydrochloride, chlorhexidine phosphanilate, chlorphensin, benzoic acid or a salt or ester thereof (e.g. sodium benzoate), propionic acid or a salt thereof, salicylic acid or a salt thereof, sorbic acid or a salt thereof (e.g. potassium sorbate), formaldehyde, paraformaldehyde, zinc pyrithione, inorganic sulphites, hydrogen sulphites, chlorobutanol, 4-hydroxybenzoic acid or a salt or ester thereof (e.g. methylparaben, ethylparaben, propylparaben), dehydroacetic acid and/or a salt thereof (e.g. sodium dehydroacetate), formic acid or a salt thereof, dibromohexamidine isethionate; thimerosal, phenylmccuric salts, undccylenic acid or a salt thereof, hexctidinc, bronopol, 5-bromo-5-nitro-1,3-dioxane, dichlorobenzyl alcohol, benzyl alcohol, triclocarban, chlororesol, triclosan, chloroxylenol, imidazolidinyl urea, polyaminopropyl biguanide, phenoxyethanol, methenamine, quaternium-15, climbazole, DMDM hydantoin, 1-hydroxy-4-methyl-6-(2,4,4-trimethylenepentyl)-2 pyridon, piroctone olamine, bromochlorophene, 0-cymen-5-ol, methylchloroisothiazolinone, methylisothiazololinone, mixtures of methylchloroisothiazolinone and methylisothiazololinone, chlorophene, chloroacetamide, phenoxyisoproponol, alkyl (C12-C22) trimethyl ammonium bromide, alkyl (C12-C22) trimethyl ammonium chloride, dimethyl oxazolidine, diazolidinyl urea, hexamidine, hexamidine diisethionate, hexamidine diparaben, hexamidine paraben, glutaral, 7-ethylbicyclooxazolidine, sodium hydroxymethylaminoacetate, silver chloride, benzethonium chloride, benzalkonium chloride, benzalkonium bromide, benzalkonium saccharinate, benzylhemiformal, iodopropynyl butylcarbamate, silver citrate, and/or combinations of two or more thereof.

The personal care composition may have a preservative system, such that the personal care composition may have a Micro Robustness Index (MRI) value of greater than 0.75 and/or an antimicrobial preservation efficacy test (APET) valuation of greater than 0.75, when in a diluted form, a non-diluted/concentrated form, or both a non-diluted/concentrated form and diluted form. In some embodiments, the personal care composition has a Micro Robustness Index value of about 0.8 or more, about 0.9 or more, about 1 or more, about 1.1 or more, about 1.2 or more, about 1.3 or more, about 1.4 or more, about 1.5 or more, about 1.7 or more, about 1.9 or more, about 2.1 or more, about 2.4 or more, or a range or a subrange formed therefrom in a diluted form, a non-diluted/concentrated form, or both a non-diluted/concentrated form and diluted form. Additionally or alternatively, the personal care composition may have a antimicrobial preservation efficacy test valuation of about 0.8 or more, about 0.9 or more, about 1 or more, about 1.1 or more, about 1.2 or more, about 1.3 or more, about 1.4 or more, about 1.5 or more, about 1.7 or more, about 1.9 or more, about 2.1 or more, about 2.4 or more, or a range or a subrange formed therefrom. In some preferred embodiments, the personal care composition has a Micro Robustness Index value and an antimicrobial preservation efficacy test valuation according to one of the respective values listed above and/or a range formed therefrom.

The personal care composition may include one or more polyol(s). Typically, the personal care compositions include the one or more polyol(s) in an amount from about 3 to about 25 wt. %, based on the total weight of the personal care composition. For example, the total amount of polyol(s) in the personal care composition may be from about 3 to about 22 wt. %, about 3 to about 20 wt. %, about 3 to about 18 wt. %, about 3 to about 16 wt. %; from about 5 to about 25 wt. %, about 5 to about 22 wt. %, about 5 to about 20 wt. %, about 5 to about 18 wt. %, about 5 to about 16 wt. %; from about 7 to about 25 wt. %, about 7 to about 22 wt. %, about 7 to about 20 wt. %, about 7 to about 18 wt. %, about 7 to about 16 wt. %; from about 9 to about 25 wt. %, about 9 to about 22 wt. %, about 9 to about 20 wt. %, about 9 to about 18 wt. %, about 9 to about 16 wt. %; from about 12 to about 25 wt. %, about 12 to about 22 wt. %, about 12 to about 20 wt. %, about 12 to about 18 wt. %, about 12 to about 16 wt. %; from about 14 to about 25 wt. %, about 14 to about 22 wt. %, about 14 to about 20 wt. %, about 14 to about 18 wt. %; from about 16 to about 25 wt. %, about 16 to about 22 wt. %, about 16 to about 20 wt. %; from about 18 to about 25 wt. %, about 21 to about 25 wt. %, or any range or subrange thereof, based on the total weight of the personal care composition.

The polyol(s) may be chosen from glycols or compounds with numerous hydroxyl groups. In some cases, the one or more polyols is/are selected from the group consisting of C₂-C₃₂ polyols. The one or more polyols may be liquid at ambient temperature (25° C.). The one or more polyols may have from 2 to 32 carbon atoms, from 3 to 16 carbon atoms, or from 3 to 12 carbon atoms.

Polyols that may be included in the personal care composition, in certain instances, include ethylene glycol, propylene glycol, butylene glycol, hexylene glycol, glycerin, diglycerin, diethylene glycol, and dipropylene glycol, and mixtures thereof. In some cases, the personal care composition comprises glycerin, and optionally one or more polyols other than glycerin. Non-limiting examples of polyols that may, optionally, be included in the personal care include and/or may be chosen from alkanediols such as glycerin, 1,2,6-hexanetriol, trimethylolpropane, ethylene glycol, propylene glycol, butylene glycol, pentylene glycol, hexylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, and a mixture thereof.

The one or more polyols may, optionally, be glycols or glycol ethers such as, e.g., monomethyl, monoethyl and monobutyl ethers of ethylene glycol, propylene glycol or ethers thereof such as, e.g., monomethyl ether of propylene glycol, butylene glycol, hexylene glycol, dipropylene glycol as well as alkyl ethers of diethylene glycol, e.g., monoethyl ether or monobutyl ether of diethylene glycol. In some cases, the one or more polyols may include or be chosen from ethylene glycol, propylene glycol, butylene glycol, hexylene glycol, pentylene glycol, 1,3-propanediol, diethylene glycol, dipropylene glycol, 1,4-butanediol, 1,5-pentanediol, hexane-1,6-diol, glycerin, diglycerin, caprylyl glycol, and/or a mixture of two or more thereof. In certain embodiments, the personal care composition may include polyol(s) selected from glycerin (preferably, vegetable refined glycerin), butylene glycol, and 1,3-propanediol, polyglutamic acid, saccharide isomerate, and combinations thereof. In some embodiments, the polyol is a polyethylene glycol, such as polyethylene glycol 600.

Additionally or alternatively, the personal care composition may include polyol(s) having a molecular weight of from about 100 to 5000 g/mol. For instance, the polyol may comprise a polyethylene glycol, a polypropylene glycol, a block polymer of polyethylene glycol and polypropylene glycol, or a combination of two or more thereof. In some embodiments, the polyol comprises a polypropylene glycol, a polypropylene glycol, and/or a block polymer of polyethylene glycol and polypropylene glycol having a molecular weight of about 100 to about 900, about 200 to about 800, about 400, about 1500 to about 2500, about 2000 to about 4500 or any range or subrange thereof.

The personal care composition may optionally include one or more thickening agent(s). The amount of thickening agent in the personal care composition, if present, may be from about 0.1 to about 7 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 7 wt. %, about 0.5 to about 5 wt. %, about 0.5 to about 4 wt. %, about 0.5 to about 3 wt. %, about 0.5 to about 2 wt. %, about 0.5 to about 1 wt. %; from about 1 to about 7 wt. %, about 1 to about 5 wt. %, about 1 to about 4 wt. %, about 1 to about 3 wt. %, about 1 to about 2 wt. %; from about 2 to about 7 wt. %, about 2 to about 5 wt. %, about 2 to about 4 wt. %, about 2 to about 3 wt. %; from about 3 to about 7 wt. %, about 3 to about 5 wt. %, about 3 to about 4 wt. %, or any range or subrange thereof, based on the total weight of the personal care composition.

Without being limited to any particular theory, the personal care compositions in some embodiments achieve preferable viscosities at least partially based on the surfactant system. For instance, the personal care compositions may obtain desirable viscosities with minimal or no thickening agents. For example, the personal care composition may include thickening agent(s) in a total amount from about 0.1 wt. %, from about 0.5 wt. %, from about 1 wt. %, from about 2 wt. %, from about 3 wt. %, or from about 3.5 wt. % up to about 1 wt. %, up to about 2 wt. %, up to about 3 wt. %, up to about 4 wt. %, or up to about 5 wt. %, including any range or subrange formed therefrom, based on the total weight of the personal care composition. In some embodiments, the personal care composition may have thickening agent(s) in a total amount of about 5 wt. % or less, about 4 wt. % or less, about 3 wt. % or less, about 2 wt. % or less, about 1 wt. % or less, about 0.5 wt. % or less, or about 0.1 wt. % or less, based on the total weight of the personal care composition.

Thickening agents may be referred to as “thickeners” or “viscosity modifying agents.” Thickening agents are typically included to increase the viscosity of the personal care compositions. Non-limiting examples of thickening agents include natural gums, polyacrylate crosspolymers or cross linked polyacrylate polymers, cationic acrylate copolymers, anionic acrylic or carboxylic acid polymers, polyacrylamide polymers, polysaccharides such as cellulose derivatives, cationic gums, polyquaterniums, vinylpyrrolidone homopolymers/copolymers, C₈₋₂₄ hydroxyl substituted aliphatic acid, C₈₋₂₄ conjugated aliphatic acid, sugar fatty esters, polyglyceryl esters, and a mixture thereof. Particular types of thickening agents that may be mentioned include the following: gums (e.g., natural gums), sucrose esters, polyvinylpyrrolidone (PVP) and copolymers, celluloses, polyquaternium compounds, and carboxylic acid or carboxylate based homopolymer or co-polymer, which can be linear or crosslinked.

The personal care composition may, in some instances, include water, e.g., in an amount from about 0.5 to about 40 wt. %, based on the total weight of the personal care composition. For example, the amount of water present in the personal care composition may be from about 0.5 to about 36 wt. %, about 0.5 to about 32 wt. %, about 0.5 to about 28 wt. %, about 0.5 to about 23 wt. %, about 0.5 to about 17 wt. %, about 0.5 to about 14 wt. %, about 0.5 to about 11 wt. %, about 0.5 to about 8 wt. %, about 0.5 to about 6 wt. %, about 0.5 to about 4 wt. %, about 0.5 to about 2 wt. %; from about 2 to about 40 wt. %, about 2 to about 36 wt. %, about 2 to about 32 wt. %, about 2 to about 28 wt. %, about 2 to about 23 wt. %, about 2 to about 17 wt. %, about 2 to about 14 wt. %, about 2 to about 11 wt. %, about 2 to about 8 wt. %, about 2 to about 6 wt. %; from about 5 to about 40 wt. %, about 5 to about 36 wt. %, about 5 to about 32 wt. %, about 5 to about 28 wt. %, about 5 to about 23 wt. %, about 5 to about 17 wt. %, about 5 to about 14 wt. %, about 5 to about 11 wt. %; from about 10 to about 40 wt. %, about 10 to about 36 wt. %, about 10 to about 32 wt. %, about 10 to about 28 wt. %, about 10 to about 23 wt. %, about 10 to about 17 wt. %; from about 15 to about 40 wt. %, about 15 to about 36 wt. %, about 15 to about 32 wt. %, about 15 to about 28 wt. %, about 15 to about 23 wt. %, from about 20 to about 40 wt. %, about 20 to about 36 wt. %, about 20 to about 32 wt. %, about 20 to about 28 wt. %; from about 25 to about 40 wt. %, about 25 to about 36 wt. %, about 25 to about 32 wt. %, or any range or subrange thereof, based on the total weight of the personal care composition. As noted above, in some embodiments, the amount of water in the personal care composition is about 4 wt. % or less, about 3 wt. % or less, about 2 wt. % or less, about 1 wt. % or less, or about 0.5 wt. % or less, based on the total weight of the personal care composition.

The personal care composition may further comprise one or more colorants. The colorants may be a pigment, a dye, or mixtures thereof. Non-limiting examples of pigments include titanium dioxide, zinc oxide, kaolin, mica etc. Non-limiting examples of dyes include food dyes suitable for food, drug and cosmetic applications, and mixtures thereof. Some color agents (colorants) are known as FD&C dyes. In some embodiments, the colorants may be present in an amount ranging from about 0.0001% wt. % to about 0.4% wt. %, including all percentages and subranges therebetween, based on the total weight of the personal care composition. In further embodiments, the colorants may be present in an amount ranging from about 0.0001% wt. % to about 4% wt. %, including all percentages and subranges therebetween, based on the total weight of the personal care composition.

The personal care composition may include one or more pH adjusters to increase or decrease the overall pH of the personal care composition. For example, one or more acids may be included to decrease the pH of the personal care composition. Examples of suitable acids for decreasing the pH of the personal care composition include, but are not limited to, citric acid, acetic acid, and the like. The personal care composition may include one or more bases, such as sodium hydroxide, potassium hydroxide and the like, to increase the pH of the personal care composition. Additional or alternative acids and bases that are suitable for adjusting the pH of the personal care composition are readily known to one of ordinary skill in the art.

The amount of the pH adjuster in the personal care composition may be based on the desired pH of the final form of personal care composition and/or product (e.g., in a non-diluted/concentrated form and/or in a diluted/ready-to-use form). For example, the total amount of the pH adjuster may range from about 0.05 to about 20 wt. %, based on the total weight of the personal care composition. In some instances, the total amount of pH adjuster is from about 0.05 to about 15 wt. %, about 0.1 to about 10 wt. %, or about 0.12 to about 5 wt. %, including ranges and sub-ranges therebetween, based on the total weight of the personal care composition.

The personal care compositions may have a pH from 4.5 to about 10, 4.5 to about 9, 4.5 to about 8, 4.5 to about 7, 4.5 to about 6; from about 5 to about 10, about 5 to about 9, about 5 to about 8, about 5 to about 7, about 5 to about 6; from about 6 to about 10, about 6 to about 9, about 6 to about 8, or about 6 to about 7; from about 7 to about 10, about 7 to about 9, or about 7 to about 8, including any ranges and subranges therebetween.

As discussed above, a personal care composition when in a diluted form (also referred herein as diluted personal care composition) and/or ready-to-use form may be produced by diluting the personal care composition with an amount of water. Although the personal care compositions are preferably diluted to produce a diluted personal care composition having about 0.3 to about 3 wt. % of a preservative system, based on the total weight of the diluted personal care composition, the personal care composition may be diluted with water at a weight ratio of about 1:0.5 to about 1:10. For instance, the personal care composition may be diluted with water at a weight ratio (personal care composition:water) of about 1:0.5 to about 1:10, about 1:0.5 to about 1:8, about 1:0.5 to about 1:6, about 1:0.5 to about 1:4, about 1:0.5 to about 1:2, about 1:0.5 to about 1:1; from about 1:1 to about 1:10, about 1:1 to about 1:8, about 1:1 to about 1:6, about 1:1 to about 1:4, about 1:1 to about 1:2; from about 1:2 to about 1:10, about 1:2 to about 1:8, about 1:2 to about 1:6, about 1:2 to about 1:4; from about 1:3 to about 1:10, about 1:3 to about 1:8, about 1:3 to about 1:6, about 1:3 to about 1:4; from about 1:4 to about 1:10, about 1:4 to about 1:8, about 1:4 to about 1:6, or any range or subrange thereof.

According to another aspect of the invention, the personal care composition may be encapsulated in a pod. The encapsulated personal care composition may include any of the components and/or ingredients described herein. The personal care composition encapsulated in the pod is typically not in a diluted form and/or is in a concentrated form.

The pod is generally formed from a film that may encapsulate and/or surround the personal care composition. In some instances, the pod is formed from two films that are coupled together, e.g., along one or more edges. When the pod is formed of two, three, or fourth films, each of the films may be the same or different.

The film is generally formed from a monomer or polymer that dissolves in water. Preferably, the film of the pod is formed of hydroxypropyl methylcellulose (HPMC), polyvinyl alcohol (PVA), or a combination of two or more thereof. In at least one embodiment, the film of the pod comprises a polymer of HPMC and PVA.

A diluted and/or ready-to-use personal care composition may be formed by combining one or more pod(s) containing the personal care composition with water to dissolve the film of the pod, thereby releasing the personal care composition. For example, the pod containing the personal care composition may be placed in a soap dispenser with water to dissolve the film of the pod and produce a diluted and/or ready-to-use form of the personal care composition.

Preferably, the film of the pod dissolves in an amount of time from about 1 second to about 60 seconds. In some embodiments, the film of the pod dissolves in an amount of time from about 1 to about 50 seconds, about 1 to about 40 seconds, about 1 to about 30 seconds, about 1 to about 25 seconds, about 1 to about 20 seconds, about 1 to about 15 seconds, about 1 to about 10 seconds; from about 3 to about 60 seconds, about 3 to about 50 seconds, about 3 to about 40 seconds, about 3 to about 30 seconds, about 3 to about 25 seconds, about 3 to about 20 seconds, about 3 to about 15 seconds, about 3 to about 10 seconds; from about 5 to about 60 seconds, about 5 to about 50 seconds, about 5 to about 40 seconds, about 5 to about 30 seconds, about 5 to about 25 seconds, about 5 to about 20 seconds, about 5 to about 15 seconds; from about 10 to about 60 seconds, about 10 to about 50 seconds, about 10 to about 40 seconds, about 10 to about 30 seconds, about 10 to about 25 seconds, about 10 to about 20 seconds; from about 15 to about 60 seconds, about 15 to about 50 seconds, about 15 to about 40 seconds, about 15 to about 30 seconds, about 15 to about 25 seconds, or any range or subrange therebetween. The amount of time for dissolving the pod may be based on agitating the water (e.g., stirring, shaking, swirling, or the like) the water and pod. In some embodiments, however, the amount of time for dissolving the pod is based on the water and pod not being agitated.

The personal care composition may be formulated such that the viscosity increases when the personal care composition is diluted with water at a weight ratio of about 1:0.5 to about 1:10. For instance, the viscosity of the personal care composition may increase when diluted with additional water at a weight ratio of about 1:0.5 to about 1:10, about 1:0.5 to about 1:8, about 1:0.5 to about 1:6, about 1:0.5 to about 1:4, about 1:0.5 to about 1:2, about 1:0.5 to about 1:1; from about 1:1 to about 1:10, about 1:1 to about 1:8, about 1:1 to about 1:6, about 1:1 to about 1:4, about 1:1 to about 1:2; from about 1:2 to about 1:10, about 1:2 to about 1:8, about 1:2 to about 1:6, about 1:2 to about 1:4; from about 1:3 to about 1:10, about 1:3 to about 1:8, about 1:3 to about 1:6, about 1:3 to about 1:4; from about 1:4 to about 1:10, about 1:4 to about 1:8, about 1:4 to about 1:6, or any range or subrange thereof.

Additionally or alternatively, the personal care composition may have a Micro Robustness Index value of greater than 0.75 (or any of the values or ranges discussed above) and/or an antimicrobial preservation efficacy test valuation of greater than 0.75 (or any of the values or ranges discussed above) when the personal care composition is diluted with water at a weight ratio of about 1:0.5 to about 1:10. For example, the personal care composition may have a Micro Robustness Index value of greater than 0.75 (or any of the values or ranges discussed above) and/or an antimicrobial preservation efficacy test valuation of greater than 0.75 (or any of the values or ranges discussed above) when diluted with additional water at a weight ratio of about 1:0.5 to about 1:10, about 1:0.5 to about 1:8, about 1:0.5 to about 1:6, about 1:0.5 to about 1:4, about 1:0.5 to about 1:2, about 1:0.5 to about 1:1; from about 1:1 to about 1:10, about 1:1 to about 1:8, about 1:1 to about 1:6, about 1:1 to about 1:4, about 1:1 to about 1:2; from about 1:2 to about 1:10, about 1:2 to about 1:8, about 1:2 to about 1:6, about 1:2 to about 1:4; from about 1:3 to about 1:10, about 1:3 to about 1:8, about 1:3 to about 1:6, about 1:3 to about 1:4; from about 1:4 to about 1:10, about 1:4 to about 1:8, about 1:4 to about 1:6, or any range or subrange thereof.

In some embodiments, the personal care composition has a weight ratio of cationic surfactant to amphoteric surfactant ranging from about 4:1 to about 1:2; optionally, from about 4:1 to about 1:1, from about 3:1 to about 1:1, or from about 2:1 to about 1:1. In other embodiments, the personal care composition has a weight ratio of cationic surfactant to non-ionic surfactant ranging from about 30:1 to about 10:1 or, optionally, from about 25:1 to about 15:1.

In certain embodiments, the personal care composition has a Micro Robustness Index value of greater than 0.75. In further embodiments, the Micro Robustness Index value of personal care composition is about 1 or more, preferably about 1.2 or more, or preferably about 1.4 or more.

In some embodiments, the personal care composition is substantially free, or free, of non-sulfate based anionic surfactants. In certain embodiments, the personal care composition is substantially free, or free, of anionic surfactants. In other embodiments, the personal care composition has less than about 1 ppm of 1,4 dioxane.

Still further embodiments of the present invention provide personal care compositions comprising: from about 30 to about 95 wt. % of a surfactant system comprising: from about 15 to about 70 wt. % of a cationic surfactant, from about 10 to about 55 wt. % an amphoteric surfactant, and a non-ionic surfactant; and a preservative system, wherein the personal care composition has a Micro Robustness Index value of greater than 0.75 upon dilution with additional water in a weight ratio of the personal care composition to additional water of from about 1:0.5 to about 1:10, and wherein all weight percentages are based on the total weight of the personal care composition.

### EXAMPLES

### Example 1

Three exemplary personal care compositions (Exs. A-C) were prepared in accordance with aspects of the invention. Exs. A-C were in the form of concentrated soap compositions. The formulations for Exs. A-C are provided in Table 1 (below).

TABLE 1

| | Ex. A | Ex. B | Ex. C |
|---|---|---|---|
| US INCI Name | (Wt. %) | (Wt. %) | (Wt. %) |
| Cetrimonium Chloride | 38.72 | 44.60 | 47.46 |
| Lauryl/myristylamidoproylamine | 20.22 | 23.38 | 24.67 |
| oxide | | | |
| Sodium Lauroamphoacetate | 10.76 | 12 | 6.52 |
| Cocamide MEA | 2.15 | 2.40 | |
| Amides, C12-18 and C18 | | | 4.96 |
| unsaturated, N-(hydroxyethyl) | | | |
| Glycerin | 7.17 | 8 | 9.96 |
| PEG-120 Methyl Glucose | 2.15 | 2.4 | 1.52 |
| Dioleate | | | |
| Benzalkonium Chloride | 2.24 | 2.5 | 1.28 |
| Sodium Benzoate, Potassium | 8.6 | | |
| Sorbate, Cetrimonium Chloride, | | | |
| and Water | | | |
| Lactic Acid | 7.17 | | |
| Hydroxyacetophenone | | 2.84 | 1.43 |
| Ethylhexylglycerin | | 0.96 | 0.48 |
| Outdoor Fizz Compact | 0.82 | 0.92 | |
| Vivacity Fragrance | | | 1.91 |

### Example 2

Ex. C was evaluated to assess the rate of dilution and the viscosity of a diluted composition prepared from Ex. C. Specifically, a sample of 46 grams of Ex. C was dissolved in water to produce a final volume of 220 mL with manual agitation. Ex. C dissolved in about 30 seconds of manual agitation. It was observed that the viscosity of the diluted composition developed once the diluted composition was homogenous, which occurred at approximately 10 minutes.

Viscosity (n) measurements were obtained with a Brookfield DV2T viscometer using a spindle number 5 at 20 RPM and a temperature of 25° C. at about 10 minutes and about 24 hours after preparing the diluted composition. The diluted composition had an initial viscosity of 3000 cPs, which is within the desired target range of 2000-4500 cPs.

### Example 3

Exs. A and B was evaluated to assess the rate of dilution. Specifically, a sample of 46 grams of Ex. A or Ex. B was dissolved in water to produce a final volume of 220 mL with manual agitation. Exs. A and B individually dissolved withing 3 to 4 minutes to produce a homogenous solution.

### Example 4

Exs. A and B were evaluated to assess the presentative systems disclosed herein. Specifically, Micro Robustness Testing (MRT) and Antimicrobial Preservation Efficacy Testing (APET) were performed on Ex. A and Ex. B. APET was performed by reconstituting Ex. A and Ex. B, separately, to a total volume of 240 mL using tap water.

Ex. A had a Micro Robustness Index (MRI) value of 1.56 and Ex. B had a MRI value of 1.34, which were both above the minimum Micro Robustness Index of 0.75. Ex. B did not meet the APET minimal criteria, while Ex. A passed the APET minimal criteria.

1. A personal care composition:
from about 30 to about 95 wt. % of a surfactant system comprising:
a cationic surfactant,
an amphoteric surfactant, and
a non-ionic surfactant; and
a preservative system comprising:
lactic acid,
benzalkonium chloride, and
at least one of sodium benzoate, potassium sorbate, and a combination of thereof,
wherein all weight percentages are based on the total weight of the personal care composition.

2. The personal care composition according to claim 1, wherein the preservative system comprising sodium benzoate.

3. The personal care composition according to claim 1, wherein the surfactant system is present in an amount from about 66 to about 95 wt. %; optionally, from about 70 to about 95 wt. %, about 75 to about 95 wt. %, or about 77 to about 95 wt. %.

4. The personal care composition according to claim 1, wherein the cationic surfactant is present in an amount from about 15 to about 70 wt. %; optionally, from about 30 to about 70 wt. %, from about 35 to about 70 wt. %, from about 38 to about 70 wt. %, from about 38 to about 60 wt. %, or from about 38 to about 50 wt. %.

5. The personal care composition according to claim 1, wherein the cationic surfactant is selected from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyl dimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, or mixtures thereof.

6. The personal care compositions of claim 1, wherein the cationic surfactant comprises cetrimonium chloride.

7. The personal care compositions of claim 1, wherein the amphoteric surfactant is present in an amount ranging from about 10 to about 55 wt. %; optionally, from about 15 to about 50 wt. %, about 20 to about 45 wt. %, about 25 to about 40 wt. %, or about 30 to about 35 wt. %.

8. The personal care compositions of claim 1, wherein the amphoteric surfactant is selected from alkyl amphoproprionates, betaines, alkyl sultaines, alkyl amphoacetates, and a combination of two or more thereof.

9. The personal care composition according to claim 1, wherein the amphoteric surfactant comprises sodium lauroamphoacetate, lauryl/myristylamidoproylamine oxide, or a combination of two or more thereof.

10. The personal care composition according to claim 1, wherein the nonionic surfactant comprises cocamide monoethanolamine (MEA), cocamide diethanolamine (DEA) or cocamide diisopropanolamine (DIPA), or a combination of two or more thereof.

11. The personal care composition according to claim 1, wherein the nonionic surfactant comprises amides, C₁₂₋₁₈ and C₁₈ unsaturated, N-(hydroxyethyl).

12. The personal care composition according to claim 1, further comprising a polyol.

13. The personal care composition according to claim 12, wherein the polyol comprises ethylene glycol, propylene glycol, butylene glycol, hexylene glycol, glycerin, diglycerin, diethylene glycol, and dipropylene glycol, or a combination of two more thereof.

14. The personal care composition according to claim 1, wherein the polyol comprises a polyethylene glycol, a polypropylene glycol, a block polymer of polyethylene glycol and polypropylene glycol, or a combination of two or more thereof.

15. A personal care composition:
from about 30 to about 95 wt. % of a surfactant system comprising:
from about 15 to about 70 wt. % of a cationic surfactant,
from about 10 to about 55 wt. % an amphoteric surfactant, and
a non-ionic surfactant; and
a preservative system comprising:
at least one of lactic acid, benzalkonium chloride, or a combination thereof, and
at least one of sodium benzoate, potassium sorbate, hydroxyacetophenone, ethylhexylglycerin, or a combination of thereof,
wherein all weight percentages are based on the total weight of the personal care composition.

16. A diluted personal care composition:
a surfactant system comprising:
a cationic surfactant,
an amphoteric surfactant, and
a non-ionic surfactant; and
from about 0.3 to about 3 wt. % of a preservative system comprising:
lactic acid,
benzalkonium chloride, and
at least one of sodium benzoate, potassium sorbate, hydroxyacetophenone, ethylhexylglycerin, and a combination of thereof,
wherein the personal care composition has a Micro Robustness Index value of greater than 0.75, and all weight percentages are based on the total weight of the diluted personal care composition.
